(19) United States
(12) Patent Application Publication
(10) Pub. No.: US 2022/0004863 A1
(43) Pub. Date: Jan. 6, 2022

(54) CONFIDENCE CLASSIFIERS FOR DIAGNOSTIC TRAINING DATA

(71) Applicant: International Business Machines Corporation, Armonk, NY (US)

(72) Inventors: Sun Young Park, San Diego, CA (US); Dustin Michael Sargent, San Diego, CA (US); David Richmond, Newton, MA (US)

(21) Appl. No.: 16/917,963

(22) Filed: Jul. 1, 2020

(51) Int. Cl. G06N 3/08; G06N 3/04; G16H 50/20; G16H 10/60; G16H 30/40

(52) U.S. Cl. CPC G06N 3/08; G06N 3/0481; G16H 30/40; G16H 10/60; G16H 50/20

(57) ABSTRACT

Automated assignment of confidence levels to medical diagnoses in a machine learning training data with respect to annotations made upon review of medical records such as x-ray films and test results. Confidence levels support machine learning for computer-aided diagnostic activity.

### BACKGROUND

The present invention relates generally to the field of machine learning, and more particularly to classifying training data according to confidence of diagnosis.

Algorithms for computer aided diagnosis are trained using annotations provided by physicians who have varying levels of expertise. The annotations are often taken from the medical reports associated with the studies. This can lead to inconsistency in the labeling of studies based on the confidence level of the physicians.

An ‘annotation” is information associated with a particular point in a document, image (such as an x-ray image), or record (such as a diagnostic report). From a cognitive perspective annotation has an important role in learning and instruction. As part of guided noticing it involves highlighting, naming, or labelling and commenting aspects of visual representations to help focus attention on specific visual aspects. In other words, it means the assignment of typological representations (culturally meaningful categories), to topological representations (for example images). This is especially important when experts, such as medical doctors, interpret visualizations in detail and explain their interpretations to others, for example by means of digital technology. Here, annotation can be a way to establish common ground between interactants with different levels of knowledge. The value of annotation has been empirically confirmed, for example, in a study which shows that in computer-based teleconsultations the integration of image annotation and speech leads to significantly improved knowledge exchange compared with the use of images and speech without annotation.”

### SUMMARY

According to an aspect of the present invention, there is a method, computer program product and/or system for assigning confidence levels to training data for machine learning models that performs the following operations (not necessarily in the following order): (i) receiving a set of medical records for a medical exam including a corresponding diagnostic annotation for each medical record; (ii) identifying a set of diagnostic activity data associated with a first diagnostic annotation of a first medical record; (iii) determining a first confidence level for the first diagnostic annotation with reference to the diagnostic activity data; and (iv) generating a set of training data from the set of medical records with diagnostic annotations and corresponding confidence level assignments including the first medical record, the first diagnostic annotation, and the first confidence level.

### BRIEF DESCRIPTION OF THE DRAWINGS

FIG. 1 is a block diagram view of a first embodiment of a system according to the present invention;

FIG. 2 is a flowchart showing a first embodiment method performed, at least in part, by the first embodiment system;

FIG. 3 is a block diagram showing a machine logic (for example, software) portion of the first embodiment system;

FIG. 4 is a system diagram showing a first embodiment method performed, at least in part, by the first embodiment system;

FIG. 5 is a system diagram showing a first embodiment method performed, at least in part, by the first embodiment system; and

FIG. 6 is a system diagram showing a first embodiment method performed, at least in part, by the first embodiment system.

### DETAILED DESCRIPTION

Some embodiments of the present invention are directed to automated assignment of confidence levels to medical diagnoses in a machine learning training data with respect to annotations made upon review of medical records such as x-ray films and test results. Confidence levels support machine learning for computer-aided diagnostic activity.

This Detailed Description section is divided into the following subsections: (i) The Hardware and Software Environment; (ii) Example Embodiment; (iii) Further Comments and/or Embodiments; and (iv) Definitions.

### I. The Hardware and Software Environment

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (for example, light pulses passing through a fiber-optic cable), or electrical signals transmitted through a wire.

A “storage device” is hereby defined to be anything made or adapted to store computer code in a manner so that the computer code can be accessed by a computer processor. A storage device typically includes a storage medium, which is the material in, or on, which the data of the computer code is stored. A single “storage device” may have: (i) multiple discrete portions that are spaced apart, or distributed (for example, a set of six solid state storage devices respectively located in six laptop computers that collectively store a single computer program); and/or (ii) may use multiple storage media (for example, a set of computer code that is partially stored in as magnetic domains in a computer's non-volatile storage and partially stored in a set of semiconductor switches in the computer's volatile memory). The term “storage medium” should be construed to cover situations where multiple different types of storage media are used.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

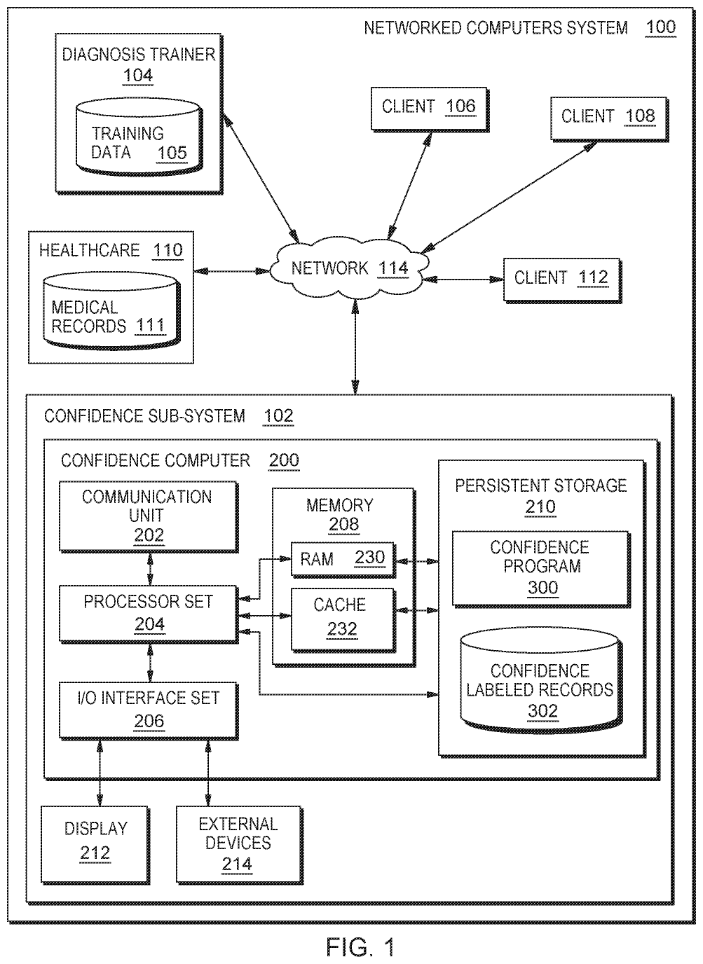

As shown in FIG. 1, networked computers system 100 is an embodiment of a hardware and software environment for use with various embodiments of the present invention. Networked computers system 100 includes: confidence subsystem 102 (sometimes herein referred to, more simply, as subsystem 102); diagnostic trainer sub-system 104, training data 105, client subsystems 106, 108, 112; healthcare sub-system 110, medical records storage 111; and communication network 114. Subsystem 102 includes: confidence computer 200; communication unit 202; processor set 204; input/output (I/O) interface set 206; memory 208; persistent storage 210; display 212; external device(s) 214; random access memory (RAM) 230; cache 232; confidence program 300; and confidence labeled records 302.

Subsystem 102 may be a laptop computer, tablet computer, netbook computer, personal computer (PC), a desktop computer, a personal digital assistant (PDA), a smart phone, or any other type of computer (see definition of “computer” in Definitions section, below). Confidence program 300 is a collection of machine readable instructions and/or data that is used to create, manage and control certain software functions that will be discussed in detail, below, in the Example Embodiment subsection of this Detailed Description section.

Subsystem 102 is capable of communicating with other computer subsystems via communication network 114. Network 114 can be, for example, a local area network (LAN), a wide area network (WAN) such as the Internet, or a combination of the two, and can include wired, wireless, or fiber optic connections. In general, network 114 can be any combination of connections and protocols that will support communications between server and client subsystems.

Subsystem 102 is shown as a block diagram with many double arrows. These double arrows (no separate reference numerals) represent a communications fabric, which provides communications between various components of subsystem 102. This communications fabric can be implemented with any architecture designed for passing data and/or control information between processors (such as microprocessors, communications and network processors, etc.), system memory, peripheral devices, and any other hardware components within a computer system. For example, the communications fabric can be implemented, at least in part, with one or more buses.

Memory 208 and persistent storage 210 are computer-readable storage media. In general, memory 208 can include any suitable volatile or non-volatile computer-readable storage media. It is further noted that, now and/or in the near future: (i) external device(s) 214 may be able to supply, some or all, memory for subsystem 102; and/or (ii) devices external to subsystem 102 may be able to provide memory for subsystem 102. Both memory 208 and persistent storage 210: (i) store data in a manner that is less transient than a signal in transit; and (ii) store data on a tangible medium (such as magnetic or optical domains). In this embodiment, memory 208 is volatile storage, while persistent storage 210 provides nonvolatile storage. The media used by persistent storage 210 may also be removable. For example, a removable hard drive may be used for persistent storage 210. Other examples include optical and magnetic disks, thumb drives, and smart cards that are inserted into a drive for transfer onto another computer-readable storage medium that is also part of persistent storage 210.

Communications unit 202 provides for communications with other data processing systems or devices external to subsystem 102. In these examples, communications unit 202 includes one or more network interface cards. Communications unit 202 may provide communications through the use of either or both physical and wireless communications links. Any software modules discussed herein may be downloaded to a persistent storage device (such as persistent storage 210) through a communications unit (such as communications unit 202).

I/O interface set 206 allows for input and output of data with other devices that may be connected locally in data communication with confidence computer 200. For example, I/O interface set 206 provides a connection to external device set 214. External device set 214 will typically include devices such as a keyboard, keypad, a touch screen, and/or some other suitable input device. External device set 214 can also include portable computer-readable storage media such as, for example, thumb drives, portable optical or magnetic disks, and memory cards. Software and data used to practice embodiments of the present invention, for example, confidence program 300, can be stored on such portable computer-readable storage media. I/O interface set 206 also connects in data communication with display 212. Display 212 is a display device that provides a mechanism to display data to a user and may be, for example, a computer monitor or a smart phone display screen.

In this embodiment, confidence program 300 is stored in persistent storage 210 for access and/or execution by one or more computer processors of processor set 204, usually through one or more memories of memory 208. It will be understood by those of skill in the art that confidence program 300 may be stored in a more highly distributed manner during its run time and/or when it is not running. Confidence program 300 may include both machine readable and performable instructions and/or substantive data (that is, the type of data stored in a database). In this particular embodiment, persistent storage 210 includes a magnetic hard disk drive. To name some possible variations, persistent storage 210 may include a solid state hard drive, a semiconductor storage device, read-only memory (ROM), erasable programmable read-only memory (EPROM), flash memory, or any other computer-readable storage media that is capable of storing program instructions or digital information.

The programs described herein are identified based upon the application for which they are implemented in a specific embodiment of the invention. However, it should be appreciated that any particular program nomenclature herein is used merely for convenience, and thus the invention should not be limited to use solely in any specific application identified and/or implied by such nomenclature.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

### II. Example Embodiment

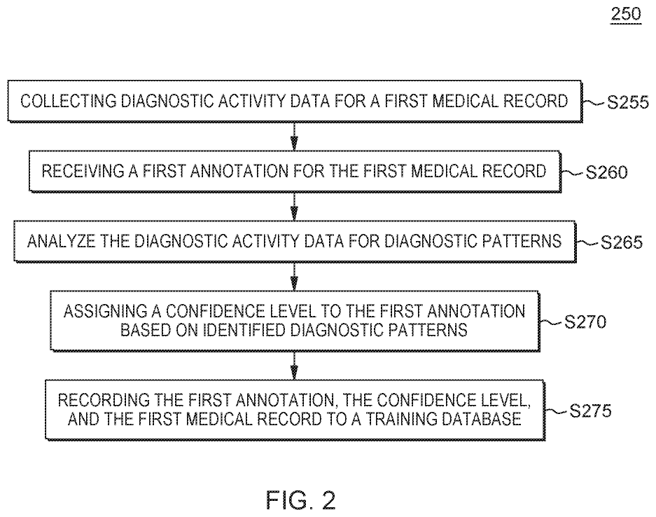

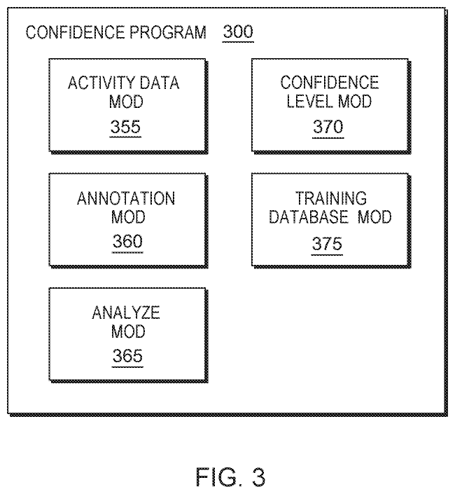

As shown in FIG. 1, networked computers system 100 is an environment in which an example method according to the present invention can be performed. As shown in FIG. 2, flowchart 250 shows an example method according to the present invention. As shown in FIG. 3, confidence program 300 performs or controls performance of at least some of the method operations of flowchart 250. This method and associated software will now be discussed, over the course of the following paragraphs, with extensive reference to the blocks of FIGS. 1, 2 and 3.

Processing begins at operation S255, where diagnostic activity mod 355 collects diagnostic activity data for a first medical record. The diagnostic activity data is associated with a diagnosing process performed by a user to diagnose the first medical record. According to some embodiments of the present invention the diagnostic activity data associated with how long and to what extent a medical record is viewed. In some embodiments of the present invention, diagnostic activity data is recorded to activity logs. Diagnostic activity includes, but is not limited to, reading exam results, use of tools or computer menus during the diagnosing process, eye tracking events while viewing a medical record image during the diagnosis process, how long the user may study or observe a computer menu, what computer tool(s) the user employed, and/or what report(s) the user reads.

Processing proceeds to operation S260, where annotation mod 360 receives a first annotation for the first medical record. The first annotation is a conclusion or comment regarding the first medical record upon performing a diagnostic process. In this example, the first annotation is a medical diagnosis and the first medical record is an x-ray image contained in medical records store 111 (FIG. 1). Alternatively, the first annotation is a diagnosis of cancer and the first medical record is a cancer screening image. Regardless of the specific purpose or type of annotation, the first annotation provides a medical diagnosis of a patient in view of images and/or documents contained in the first medical record. Additionally, other written notes and/or oral comments recorded by the diagnosing user may be used by embodiments of the present invention to support a determination of confidence level of annotations associated with a diagnosis.

Processing proceeds to operation S265, where analyze mod 365 analyzes the diagnostic activity data for diagnostic patterns. Diagnostic patterns may be found in training data providing support for determinations of confidence levels for the corresponding annotations for medical records. In this example, the training data is obtained from training data store 105 of diagnosis trainer module 104 (FIG. 1). The training data includes records having pre-assigned confidence levels for various annotations made and corresponding diagnostic activity data. Alternatively, diagnostic patterns are identified in the diagnostic activity data without regard to particular patterns found in training data. Patterns, whether exhibited during the viewing of the medical record or the use of computer tools, may be identified in a lookup table including standardized diagnostic patterns of interest including durations of exams, examination of particular records, and behavior, such as eye movement, during the diagnosis.

Processing proceeds to operation S270, where confidence level mod 370 assigns a confidence level to the first annotation based on identified diagnostic patterns. In this example, confidence levels are assigned according to the assigned confidence levels in the training data exhibiting similar diagnostic patterns. In this example, the medical records having assigned confidence levels are stored in confidence labeled records store 302 (FIG. 1). Where different confidence levels are associated with different diagnostic patterns a combination of confidence levels is taken into consideration. In this example, the highest confidence level exhibited in the combination is the assigned confidence level. Alternatively, the confidence level assigned in an average confidence level of the combination identified. Alternatively, the confidence level for each pattern is identified in a lookup table where each pre-defined pattern has a corresponding confidence level. Alternatively, the most frequently appearing confidence level associated with the identified patterns in the assigned confidence level. Alternatively, where two or more confidence levels appear an equal number of times as the most frequently appearing confidence level, an average of those confidence levels is selected. Alternatively, where two or more confidence levels appear an equal number of times as the most frequently appearing confidence level, the highest confidence level of the two or more confidence levels is the assigned confidence level.

Processing ends with operation S275, where training database mod 375 records the first annotation, the confidence level, and the first medical record to a training database. In this example, the information is recorded to training data store 105 (FIG. 1). Alternatively, medical records for which confidence levels are assigned and stored, for example in confidence labeled records store 302, are made available for use in and support of additional confidence level determinations. Alternatively, confidence levels are shared with healthcare providers to support development of a wellness plan for the patient associated with the first medical record.

### III. Further Comments and/or Embodiments

Some embodiments of the present invention recognize the following facts, potential problems and/or potential areas for improvement with respect to the current state of the art: (i) medical reports do not contain measures of confidence in the physicians' diagnosis; (ii) if physicians' confidence scores were available, such scores could improve the training of computer aided diagnosis algorithms; (iii) a physicians' confidence does not currently use a machine learning approach to automatically estimate the physician's confidence based on their interaction with medical report studies; and (iv) existing machine learning systems for medical imaging that incorporate estimation of physician confidence, to improve the training and inference accuracy of CAD algorithms, are not known to exist.

Some embodiments of the present invention may include one, or more, of the following operations, features, characteristics and/or advantages: (i) training an algorithm to estimate a physicians' confidence in their diagnosis of a patient for a particular type of medical exam, for example, mammography for breast cancer screening; (ii) using the estimates of physician confidence to improve the training of computer aided diagnosis (CAD) algorithms for the same type of medical exam; and (iii) allows CAD algorithms to be trained to simulate physicians, by minimizing errors on “easy” cases before moving on to learning more difficult concepts.

Some embodiments of the present invention apply a custom loss function that incorporates multiple annotations and/or confidence measurements. That is, the loss function is defined based on measures including: (i) agreement between the annotators; and/or (ii) the confidence rating for each annotation. For example, for computer-aided detection of breast density, categories A, B, C, and D may apply. By denoting the confidence-weighted sum of confidence levels with a four-vector term ρₖⁱ for each category k∈{A B C D}, the confidence-weighted sum is:

$$ρ \frac{i}{k} = {\sum }_{j = 1}^{J} {c}^{i , j} {a}_{k}^{i , j} ,$$

where agreement between the annotators is a with annotator j of case i of the training data and annotation confidence is c for the case i with respect to the annotator j.

In that way, annotations of medium confidence only count as half credit and low confidence annotations are thrown away. By normalizing the vector, a weight vector ωₖⁱ for each class k and each case i is written as follows:

$${ω}_{k}^{i} = \frac{1}{{\sum }_{i ∈ \{ ABCD \} ρ \frac{i}{l}}} ρ \frac{i}{k}$$

The loss L′ for case i is obtained by the weighted sum of the error for each category as described below:

$${L}^{i} = - {\sum }_{k ∈ \{ ABCD \}} {ω}^{\frac{i}{k}} log ( σ \frac{i}{k} ) ,$$

where σₖⁱ is the predicted probability from the algorithm that case i has density category k.A method according to an embodiment of the present invention for training an algorithm to estimate physician confidence includes the following operations (not necessarily in the following order): (i) when a machine learning algorithm is trained for computer aided diagnosis, it typically uses labels for each training example provided by physicians (no finding, benign, malignant, suspicious, etc.); (ii) the labels provide the classification of each training example, but do not include a measurement of the confidence in the assessment or diagnosis; and (iii) given a subset of the training examples that also include radiologist confidence scores along with logs of their activities while viewing the exam factor in: (a) activity logs or diagnostic activity data that could include factors such as how long they spent reading the exam, what tools were used/actions were performed, the contents of any audio recordings made by the physician, the contents of medical reports, or eye tracking data while viewing the exam, etc., (b) confidence scores that could be provided manually by radiologists, (c) alternatively, if CAD algorithms for the task already exist, their agreement among themselves and with the medical report, along with the probabilities returned by the CAD algorithms, could be used to automatically assign confidence scores to each study, and (d) a machine learning algorithm could be trained, either using traditional or deep learning, to take a representation of the radiologists' activity when viewing an exam and estimate their confidence in the resulting classification.

A method according to an embodiment of the present invention for using the confidence estimates to train CAD algorithms includes the following operations (not necessarily in the following order): (i) given the above algorithm, all of the training data could be analyzed and assigned a physician confidence score; and (ii) multiple strategies could be adopted to improve CAD algorithm training using these scores including: (a) the training loss function could weight examples by confidence, given a higher weight to classification errors on cases with high confidence scores and vice versa, (b) an adjusted learning schedule could be adopted, by scheduling batches and adjusting batch size so that the algorithm starts off by learning the high confidence cases and then proceeds to the more difficult, low confidence cases, and (c) the algorithm could be trained normally and the confidence estimates used to adjust the algorithms' output at inference time.

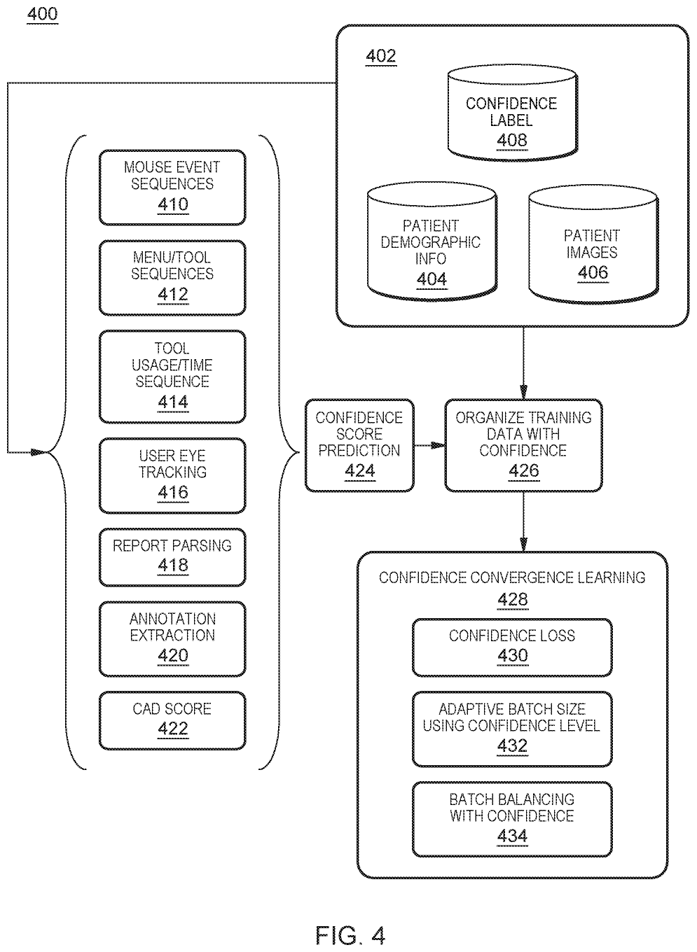

As shown in FIG. 4, system diagram 400 for a confidence learning system includes: data storage block 402; patient demographic info data storage 404; patient images data storage 406; confidence label data storage 408; mouse event sequences block 410; menu/tool sequences block 412; tool usage/time sequence block 414; user eye tracking block 416; report parsing module 418; annotation extraction module 420; CAD score block 422; confidence score prediction block 424; organize training data with confidence block 426; confidence convergence learning program 428; confidence loss block 430; adaptive batch size using confidence level block 432; and batch balancing with confidence block 434.

Some embodiments of the present invention learn to supplement annotations such as no finding, benign, malignant, and suspicious with user-specific labels indicating confidence of the user in recording each annotation for medical images. The learning is performed with a set of annotation and confidence-rated images, stored in patient images store 406 and with corresponding confidence labels stored in confidence label store 408. Further, according to FIG. 4, patient images store 406 includes images having annotations assigned by a user, such as a physician or radiologist. Annotation extraction mod 420 identifies annotations made with respect to individual images for a particular patient with support of report parsing mod 418. Other modules collect data associated with diagnostic activities of the user who annotates each image, for example with eye tracking software and input data from mouse, menu, and tool usage and/or sequences. Further, according to some embodiments of the present invention, the report parsing mod parses prior examination reports of the user in support of estimating the confidence of the annotation made to the current exam image(s) Subsequently, confidence score prediction module 424 makes a prediction of the confidence in the annotation based upon the various inputs.

When training a system on annotated and confidence rated data, organize training data module 426 compares predicted confidence scores to recorded confidence scores in confidence label store 408. Confidence convergence learning program 428 processes the training data for calibrating and further training of the system via confidence loss module 430, adaptive batch size module 432, and batch balancing module 434.

Some embodiments of the present invention recognize that confidence loss controls the degree to which training focuses attention on easy (high confidence) examples versus difficult (low confidence) examples. The parameters of the confidence loss function can be tuned to reduce the impact of low confidence examples that may be noisy and confuse the training process. On the other hand, the parameters can also be tuned to focus training on the hard examples and avoid becoming dominated by easy examples, to increase the discrimination capabilities of the model.

Some embodiments of the present invention recognize that adaptive batch size and batch balancing help to speed up the training while attempting to avoid local minima. Adaptive batch size increases the size of batches for difficult (low confidence) examples and reduces batch size for easy examples. This has the effect of changing the learning rate adaptively, that is training takes smaller steps based on more samples when examples are difficult and larger steps when easy examples are seen. Taking large steps on easy examples allows training to converge faster, while using small batch sizes on easy examples slightly randomizes the search direction, which helps avoid local minima. Adaptive batch balancing also helps to avoid local minima by making sure that each batch has a non-uniform distribution of examples. Including examples from multiple vendors and demographics in each batch, especially early in training, prevents training from being locked into a sub-optimal region of the loss function caused by focusing too much on specific characteristics of the training examples.

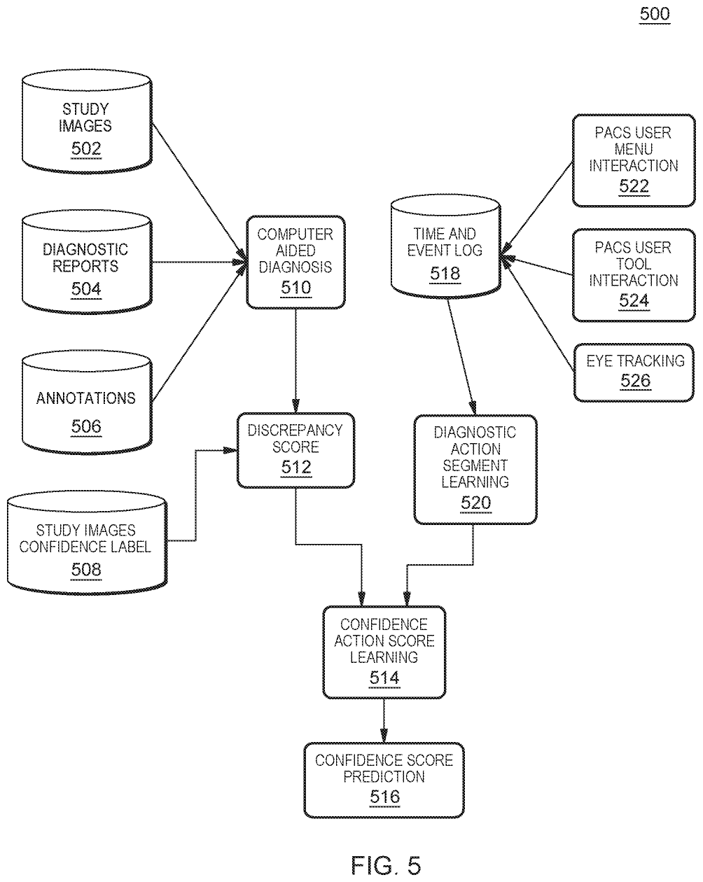

As shown in FIG. 5, system diagram 500 for confidence score prediction includes study images data storage 502; diagnostic reports data storage 504; annotations data storage 506; study images confidence label data storage 508; computer aided diagnosis block 510; discrepancy score block 512; confidence action score learning block 514; confidence score prediction block 516; time and event log data storage 518; diagnostic action segment learning block 520; PACS (picture archiving and communication system) user menu interaction block 522; PACS user tool interaction block 524; and eye tracking block 526.

Some embodiments of the present invention use trained prediction systems to predict confidence scores for a batch of annotated images and/or medical reports where diagnostic activity of the annotator is collected. FIG. 5 illustrates predicted confidence scores according to some embodiments of the present invention where a trained prediction system is deployed. Diagnostic data is collected by computer aided diagnosis mod 510 while corresponding diagnostic action data is processed by diagnostic action mod 520. Confidence action score mod 514 associates the diagnostic reports with diagnostic actions to support a prediction of confidence provided by confidence score module 516.

Some embodiments of the present invention further apply confidence labels produced by a user performing the diagnosis, such as may be stored in study images store 508. For such embodiments, the confidence labels may be retrieved by discrepancy score module 512 and made available to confidence action score mod 514.

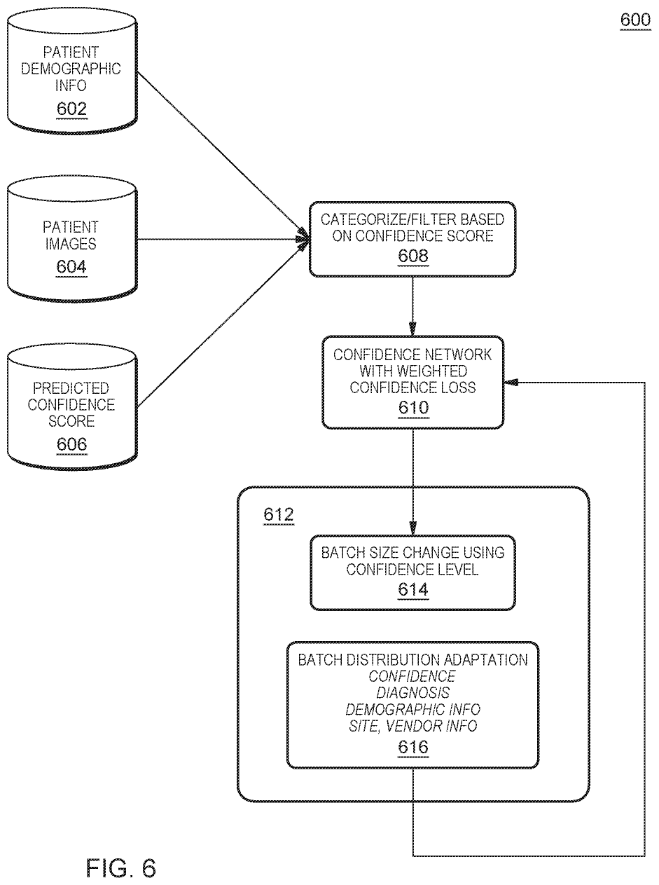

As shown in FIG. 6, system diagram 600 for diagnosis system having confidence ratings includes: patient demographics info data storage 602; patient images data storage 604; predicted confidence score data storage 606; categorize/filter based on confidence score block 608; confidence network with weighted confidence loss block 610; refinement program 612; batch size change using confidence level block 614; and batch distribution adaptation block 616, which includes the following parameters: (i) confidence; (ii) diagnosis; (iii) demographic info; and (iv) site, vendor info.

Some embodiments of the present invention assign predicted diagnostic confidence to medical records as illustrated in FIG. 6. Predicted confidence scores (e.g. confidence score database 606 and corresponding medical records, such as found in patient images store 604, are processed for weighting by categorize and filter module 608. The categorized records are assigned a weighted confidence loss by confidence network module 610 prior to refining the predicted confidence level via refinement program 612.

According to some embodiments of the present invention confidence level block 614 and batch distribution adaptation block 616 are implemented in the following way. It should be noted that this is one example implementation illustrating general operations when practicing embodiments of the present invention. For the confidence level block, assume there is a default batch size, for example 16. Choose 16 random examples from the training data for the batch and check the confidence levels of the 16 examples. If some threshold percentage of them are low confidence, such as 50%, increase the batch size to 32 and add 16 more (mostly low confidence) examples to the batch.

For the batch distribution adaptation block, assume a batch size of 16, and a training set with three vendors, vendor A (50%), B (30%), and C (20%). Start adding examples to the batch by random selection, keeping track of how many of each vendor have been added. If the number of vendor A examples exceeds a specified percentage, for example 50%, reduce the probability of selecting vendor A examples. This way each batch will not have the exact same distribution but will have some vendor variety in each batch.

Some embodiments of the present invention begin with a desired distribution of vendor A, B, and C for each batch, and add exactly that number of cases from each vendor to each batch. For the above example, one would select 8 A, 5, B, and 3 C for each batch. This could also be done with characteristics other than vendor such as demographics.

As a use case/example, a method according to an embodiment of the present invention for training a CAD algorithm to diagnose breast cancer from mammography exams includes the following operations (not necessarily in the following order): (i) given: training, validation, and test sets of mammography exams from several hospitals with medical reports indicating the radiologist's diagnosis of each case (dataset A); (ii) given: a subset of dataset A that has been annotated by the reading physicians with a measure of their confidence in the diagnosis for each study, that is, a low, medium, high or a percentage rating (dataset B); and (iii) optional: if there is a CAD algorithm for mammography already available, confidence annotations could be provided automatically for some studies.

Some embodiments of the present invention may include one, or more, of the following operations, features, characteristics and/or advantages: (i) train a CNN (convolutional neural network) (CNN-B) that takes the images in a mammography study and assigns them a radiologist's confidence rating according to the chosen measure (training done with dataset B); (ii) design a CNN (CNN-A) to classify breast cancer from the images in a mammography study, for example in the category 6 BI-RAD (breast imaging reporting and data system) where proven malignancy has been determined; (iii) train CNN-A on dataset A, but modify the training using CNN-B which includes: (a) assigning a confidence rating to each of the training exams in dataset A using CNN-B, (b) modifying the loss function for the optimizer for CNN-A to weight each training example by physician confidence, higher weights for higher confidence scores, to ensure that CNN-A does not make errors on the easy cases, (c) scheduling the batch generation for training CNN-A to present the training examples in decreasing order of confidence, again ensuring that CNN-A learns the easy cases before moving on to difficult cases, and (d) optional: after CNN-A is trained, modify its inference behavior by first assigning a confidence label to the study using CNN-B, and then adjusting the label assigned by CNN-A according to the confidence estimate; and (iv) the same methods could be used for any computer-aided diagnosis algorithm, for example for liver cancer, prostate cancer, etc.

Some embodiments of the present invention may include one, or more, of the following operations, features, characteristics and/or advantages: (i) confidence learning; (ii) confidence training; (iii) confidence annotation; and (iv) confidence estimation.

A method for training a model with a confidence associated with training data, according to an embodiment of the present invention, includes the following operations (not necessarily in the following order): (i) receives training data for training an artificial intelligence (AI) machine learning (ML) model; (ii) applies a confidence assessment to the training data; (iii) trains the model utilizing a weighting based on the confidence assessment; (iv) wherein the confidence assessment is received as input (annotations) associated with the training data; (v) wherein the confidence assessment is determined based on confidence scores associated with a classification algorithm associated with an assessment of features identified in the training data; (vi) wherein the assessment of features related to a diagnostic finding in images and the confidence assessment is determined based on identified activities associated with a diagnosing user viewing an image such as duration of viewing period, number of times the image is viewed during the viewing period, and duration of individual viewing moments making up the viewing period; and (vii) wherein the classifications are for a mammogram for breast cancer screening and the identified features are selected from a group consisting of no finding, benign, malignant, and suspicious.

Some embodiments of the present invention may include one, or more, of the following operations, features, characteristics and/or advantages: (i) estimates the physicians' confidence in their diagnosis; (ii) uses the confidence of the annotators; (iii) creates a classifier that automatically assesses an annotator's confidence based on their actions during annotation; (iv) does not need a very large set of confidence-annotated training samples; (v) uses a deep learning model; (vi) uses the annotation disagreement to affect the training schedule other than the loss function; (vii) creates a classifier to predict annotator confidence; (viii) modifies the training schedule based on the confidence; (ix) predicts annotator confidence from their actions while performing annotations; and (x) modifies batch scheduling or loss function.

### IV. Definitions

Present invention: should not be taken as an absolute indication that the subject matter described by the term “present invention” is covered by either the claims as they are filed, or by the claims that may eventually issue after patent prosecution; while the term “present invention” is used to help the reader to get a general feel for which disclosures herein are believed to potentially be new, this understanding, as indicated by use of the term “present invention,” is tentative and provisional and subject to change over the course of patent prosecution as relevant information is developed and as the claims are potentially amended.

Embodiment: see definition of “present invention” above—similar cautions apply to the term “embodiment.”

and/or: inclusive or; for example, A, B “and/or” C means that at least one of A or B or C is true and applicable.

Including/include/includes: unless otherwise explicitly noted, means “including but not necessarily limited to.”

Module/Sub-Module: any set of hardware, firmware and/or software that operatively works to do some kind of function, without regard to whether the module is: (i) in a single local proximity; (ii) distributed over a wide area; (iii) in a single proximity within a larger piece of software code; (iv) located within a single piece of software code; (v) located in a single storage device, memory or medium; (vi) mechanically connected; (vii) electrically connected; and/or (viii) connected in data communication.

Computer: any device with significant data processing and/or machine readable instruction reading capabilities including, but not limited to: desktop computers, mainframe computers, laptop computers, field-programmable gate array (FPGA) based devices, smart phones, personal digital assistants (PDAs), body-mounted or inserted computers, embedded device style computers, application-specific integrated circuit (ASIC) based devices.

What is claimed is:

1. A computer-implemented method for assigning confidence levels to training data for machine learning models comprising:
receiving a set of medical records for a medical exam including a corresponding diagnostic annotation for each medical record;
identifying a set of diagnostic activity data associated with a first diagnostic annotation of a first medical record;
determining a first confidence level for the first diagnostic annotation with reference to the diagnostic activity data; and
generating a set of training data from the set of medical records with diagnostic annotations and corresponding confidence level assignments including the first medical record, the first diagnostic annotation, and the first confidence level.

2. The computer-implemented method of claim 1 further comprising:
weighting the diagnostic annotations of the medical diagnosis according to the determined confidence levels; and
wherein:
the set of training data includes the weighted diagnostic annotations.

3. The computer-implemented method of claim 1 further comprising:
training a convolutional neural network with the weighted diagnoses as part of the modified training data.

4. The computer-implemented method of claim 1, wherein:
the set of medical records includes images generated during the medical exam; and
the set of diagnostic activity data for a medical record includes how long an annotator viewed an image of the medical record when annotating the image.

5. The computer-implemented method of claim 1, wherein:
the medical exam is a breast cancer screening;
the image is a mammogram; and
the annotation is a member selected from the group consisting of:
a) no finding,
b) benign,
c) malignant, and
d) suspicious.

6. The computer-implemented method of claim 1 further comprising:
using the set of training data to train CAD (computer-aided diagnosis) algorithms to:
use a training loss function to weight examples by confidence, giving a higher weight to classification errors on cases with high confidence scores and vice versa;
adapt an adjusted learning schedule by scheduling batches and adjusting batch size so that the algorithm starts off by learning the high confidence cases and then proceed to the more difficult, low confidence cases;
train the algorithm without confidence adjustment; and
adjusting an output of the algorithm based on the confidence estimates.

7. A computer program product comprising:
a set of storage device(s); and
computer code stored collectively in the set of storage device(s), with the computer code including data and instructions to cause a processor(s) set to perform at least the following operations:
receiving a set of medical records for a medical exam including a corresponding diagnostic annotation for each medical record,
identifying a set of diagnostic activity data associated with a first diagnostic annotation of a first medical record,
determining a first confidence level for the first diagnostic annotation with reference to the diagnostic activity data, and
generating a set of training data from the set of medical records with diagnostic annotations and corresponding confidence level assignments including the first medical record, the first diagnostic annotation, and the first confidence level.

8. The computer program product of claim 7 wherein the computer code further includes instructions for causing the processor(s) set to perform the following operation(s):
weighting the diagnostic annotations of the medical diagnosis according to the determined confidence levels; and
wherein:
the set of training data includes the weighted diagnostic annotations.

9. The computer program product of claim 7 further comprising:
training a convolutional neural network with the weighted diagnoses as part of the modified training data.

10. The computer program product of claim 7, wherein:
the set of medical records includes images generated during the medical exam; and
the set of diagnostic activity data for a medical record includes how long an annotator viewed an image of the medical record when annotating the image.

11. The computer program product of claim 7, wherein:
the medical exam is a breast cancer screening;
the image is a mammogram; and
the annotation is a member selected from the group consisting of:
a) no finding,
b) benign,
c) malignant, and
d) suspicious.

12. The computer program product of claim 7 wherein the computer code further includes instructions for causing the processor(s) set to perform the following operation(s):
using the set of training data to train CAD (computer-aided diagnosis) algorithms to:
use a training loss function to weight examples by confidence, giving a higher weight to classification errors on cases with high confidence scores and vice versa;
adapt an adjusted learning schedule by scheduling batches and adjusting batch size so that the algorithm starts off by learning the high confidence cases and then proceed to the more difficult, low confidence cases;
train the algorithm without confidence adjustment; and
adjusting an output of the algorithm based on the confidence estimates.

13. A computer system comprising:
a processor(s) set;
a set of storage device(s); and
computer code stored collectively in the set of storage device(s), with the computer code including data and instructions to cause the processor(s) set to perform at least the following operations:
receiving a set of medical records for a medical exam including a corresponding diagnostic annotation for each medical record,
identifying a set of diagnostic activity data associated with a first diagnostic annotation of a first medical record,
determining a first confidence level for the first diagnostic annotation with reference to the diagnostic activity data, and
generating a set of training data from the set of medical records with diagnostic annotations and corresponding confidence level assignments including the first medical record, the first diagnostic annotation, and the first confidence level.

14. The computer system of claim 13 wherein the computer code further includes instructions for causing the processor(s) set to perform the following operation(s):
weighting the diagnostic annotations of the medical diagnosis according to the determined confidence levels; and
wherein:
the set of training data includes the weighted diagnostic annotations.

15. The computer system of claim 13 further comprising:
training a convolutional neural network with the weighted diagnoses as part of the modified training data.

16. The computer system of claim 13, wherein:
the set of medical records includes images generated during the medical exam; and
the set of diagnostic activity data for a medical record includes how long an annotator viewed an image of the medical record when annotating the image.

17. The computer system of claim 13, wherein:
the medical exam is a breast cancer screening;
the image is a mammogram; and
the annotation is a member selected from the group consisting of:
a) no finding,
b) benign,
c) malignant, and
d) suspicious.

18. The computer system of claim 13 wherein the computer code further includes instructions for causing the processor(s) set to perform the following operation(s):
using the set of training data to train CAD (computer-aided diagnosis) algorithms to:
use a training loss function to weight examples by confidence, giving a higher weight to classification errors on cases with high confidence scores and vice versa;
adapt an adjusted learning schedule by scheduling batches and adjusting batch size so that the algorithm starts off by learning the high confidence cases and then proceed to the more difficult, low confidence cases;
train the algorithm without confidence adjustment; and
adjusting an output of the algorithm based on the confidence estimates.
